(19) United States
(12) Patent Application Publication
(10) Pub. No.: US 2023/0218538 A1
(43) Pub. Date: Jul. 13, 2023

(54) TRANSDERMAL PATCH

(71) Applicant: BUZZZ PHARMACEUTICALS LIMITED, Raheny (IE)

(72) Inventor: Kirti VALIA, East Windsor, NJ (US)

(21) Appl. No.: 18/185,175

(22) Filed: Mar. 16, 2023

Related U.S. Application Data
(63) Continuation of application No. 16/070,576, filed on Jul. 17, 2018, now abandoned.

(30) Foreign Application Priority Data
Jan. 18, 2016 (GB) 1600918.5

(51) Int. Cl. A61K 9/70; A61K 31/485; A61K 47/12

(52) U.S. Cl. CPC A61K 9/7061; A61K 31/485; A61K 47/12

(57) ABSTRACT

The present invention relates to a transdermal patch for the for the transdermal administration of oxymorphone. The present invention also relates to processes for the preparation of the transdermal patches defined herein, as well as to the use of these patches for the treatment of pain.

### CROSS-REFERENCE TO RELATED APPLICATIONS

This application is a continuation of U.S. application Ser. No. 16/070,576, filed Jul. 17, 2018, which is a U.S. national phase of PCT/EP2017/051007, filed on Jan. 18, 2017, which claims priority to United Kingdom Patent Application No. 1600918.5, filed on Jan. 18, 2016, the disclosure of each of which is hereby incorporated herein by reference in its entirety.

### FIELD OF THE INVENTION

The present invention relates to a transdermal patch. More specifically, the present invention relates to a transdermal patch for the transdermal administration of oxymorphone. The present invention also relates to processes for the preparation of the transdermal patches defined herein, as well as to the use of these patches for the treatment of pain.

### BACKGROUND OF THE INVENTION

Opioid analgesics are widely used in the clinic to treat moderate to severe pain. However, despite their clinical efficacy, opioid analgesics do suffer from some major drawbacks. One major drawback is that prolonged opioid analgesic use can lead to dependence, which gives rise to withdrawal symptoms if the opioid analgesic treatment is stopped abruptly. This opioid dependence can make opioid analgesics very addictive and prone to abuse. In addition, opioid analgesics are also well known for their ability to produce a feeling of euphoria, motivating some to use opioids recreationally.

The prevalence of opioid analgesic abuse is a major problem and the Food and Drug Administration (FDA) in the United States has initiated a program to encourage manufacturers of extended release and transdermal opioid formulations to consider innovative strategies to reduce the risk of abuse, and thereby encourage safe opioid use.

The transdermal delivery of opioid analgesics is a convenient and effective way to deliver opioid analgesics. However, there remains a need for improved approaches for the transdermal delivery of opioid analgesics.

Aspects of the invention were devised with the foregoing in mind.

### SUMMARY OF THE INVENTION

In a first aspect, there is provided a transdermal patch comprising:
1-6% w/w of oxymorphone, or a pharmaceutically acceptable salt thereof;
82-97% w/w of a pharmaceutically acceptable pressure sensitive polyacrylate adhesive comprising either a plurality of hydroxyl functional groups or no functional groups; and
2-12% w/w of a penetration enhancer selected from oleic acid or linoleic acid.

The transdermal patches of the present invention are able to provide excellent average fluxes of oxymorphone through the skin, despite the total concentration of oxymorphone present in the transdermal patch being low. This excellent level of oxymorphone flux at low concentrations renders the transdermal patches of the present invention particularly well suited for the treatment of pain, whilst rendering their susceptibility to abuse low.

In a second aspect, the present invention provides a transdermal patch as herein defined for use in therapy.

In another aspect, the present invention provides a transdermal patch as herein defined for use in the treatment of a condition selected form the group consisting of opioid dependence, alcohol dependence, polydrug addiction, pain, cocaine addiction, eating disorders (e.g., binge eating) and treatment-resistant depression.

In another aspect, the present invention provides a method of treating a condition selected form the group consisting of opioid dependence, alcohol dependence, polydrug addiction, pain, cocaine addiction, eating disorders (e.g., binge eating) and treatment-resistant depression in a subject in need of such treatment, said method comprising administering a transdermal patch as defined herein.

### DETAILED DESCRIPTION OF THE INVENTION

### Transdermal Patch of the First Aspect of the Invention

As indicated above, in a first aspect, the present invention provides a transdermal patch comprising an oxymorphone layer that comprises:
1-6% w/w of oxymorphone, or a pharmaceutically acceptable salt thereof;
82-97% w/w of a pharmaceutically acceptable pressure sensitive polyacrylate adhesive comprising either a plurality of hydroxyl functional groups or no functional groups; and
2-12% w/w of a penetration enhancer selected from oleic acid or linoleic acid.

The oxymorphone layer of the transdermal patch of the present invention has a first surface that contacts a backing film (as described further herein) and a second opposing surface that contacts the skin during use. The oxymorphone present in the oxymorphone layer of the transdermal patch diffuses or permeates into the skin over time to provide the desired analgesic effect.

The second surface of the transdermal patch is suitably covered with a peelable release liner that extends across the entire second surface of the transdermal patch, but which can be removed to expose the second surface of the transdermal patch prior to application of the patch to the skin.

### Backing Film

Suitably the backing film is occlusive. The backing film may be of any thickness, but is suitably between about 0.1 to 100 mil thick. Suitable materials include, but are not limited to, synthetic polymers including, for example, polyesters, polycarbonates, polyimides, polyethylene, poly(ethylene terphthalate), polypropylene, polyurethanes and polyvinylchlorides. The backing film may also be a laminate comprising additional layers that may include vapour deposited metal, such as aluminium, additional synthetic polymers, and other materials, to enable a heat seal, such as EVA copolymer. Suitably, the backing film comprises occlusive Scotchpak 9730®, Scotchpak 9732® or Scotchpak 9733® obtainable from 3M. Most suitably, the backing film comprises Scotchpak 9733®.

In one embodiment, the backing film has a thickness of 0.1 to 50 mil (2.5 μm to 1.25 mm), more suitably, 1-20 mil (25 μm to 0.635 mm), even more suitably 1-10 mil (25 μm to 0.25 mm), and most suitably 1-5 mil (25 μm to 127 μm).

### Release Liner

The release liner is typically disposed on an opposite surface of the multi-laminate (i.e. the second surface of the oxymorphone layer) to the backing film and provides a removable protective or impermeable layer, usually but not necessarily rendered non-stick so as to not adhere to the oxymorphone layer. The release liner serves to protect the oxymorphone layer during storage and transit, and is intended to be removed prior to application to the skin. The release liner may be formed from the same materials used for the backing membrane, but may be formed from metal foils, Mylar®, polyethylene terephthalate, siliconized polyester, fumed silica in silicone rubber, polytetrafluoroethylene, cellophane, siliconized paper, aluminized paper, polyvinyl chloride film, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene.

Suitably, the release liner is an occlusive or semi-occlusive backing film being compatible with the pharmaceutically-acceptable adhesive present in the pharmaceutical formulation layer.

Suitably, the release liner may be selected from Scotchpak 9741®, Scotchpak 1022®, Scotchpak 9742®, Scotchpak 9744®, Scotchpak 9748® and Scotchpak 9755®, all of which are obtainable from 3M and comprise fluoropolymers coated onto polypropylene or polyester film. Other suitable release liners made by other manufacturers may also be used. The release liner may be of any thickness known in the art. Suitably the release liner has a thickness of about 0.01 mm to about 2 mm.

In one embodiment, the release liner is selected from Scotchpak 9741®, Scotchpak 1022® or Scotchpak 9744®. Suitably, the release liner is Scotchpak 9744®.

In another embodiment, the release liner has a thickness of 0.1 to 50 mil (2.5 μm to 1.25 mm), more suitably, 1-20 mil (25 μm to 0.635 mm), even more suitably 1-10 mil (25 μm to 0.25 mm), and most suitably 1-5 mil (2.5 μm to 127 μm).

The container or closure system may be made from a range of materials suitable for protecting the packaged transdermal patch from moisture and light.

### Transdermal Patch

In one embodiment, the oxymorphone layer has a dry thickness of 0.1-100 mil (2.5 μm to 2.5 mm), suitably, 1-50 mil (25 μm to 1.25 mm), more suitably 2-20 mil (50 μm to 0.635 mm), yet more suitably 5-20 mil (0.125 mm to 0.635 mm), even more suitably 10-15 mil (0.25 mm to 0.38 mm) and most suitably, 10-12 mil (0.25 mm to 0.305 mm).

In an embodiment, the oxymorphone layer has a dry thickness of 1 to 15 mil, 1 to 12 mil, 10 to 12 mil, 2 to 3 mil or 2.5 to 3 mil.

### Oxymorphone

The transdermal patch of the present invention comprises oxymorphone.

The amount of oxymorphone present in the transdermal patch of the present invention will depend on how soluble it is in the pharmaceutically-acceptable adhesive and excipients present in this layer and how much of the oxymorphone is required in order to achieve the desired therapeutic effect. Typically, the oxymorphone will be present at an amount of 1-10% w/w in the oxymorphone layer.

In one embodiment, the amount of oxymorphone present is 1-6% w/w in the oxymorphone layer of the transdermal patch. Suitably, the amount of oxymorphone present is 3-5% w/w, more suitably 2-4% w/w, and even more suitably about 2.5-3% w/w in the oxymorphone layer of the transdermal patch. In an embodiment, the amount of oxymorphone present is 2.0-3.0% w/w in the oxymorphone layer of the transdermal patch. In a further embodiment, the amount of oxymorphone present is 2.2-2.8% w/w in the oxymorphone layer of the transdermal patch. In yet another embodiment, the amount of oxymorphone present is 2-2.6% w/w in the oxymorphone layer of the transdermal patch. For example, the oxymorphone layer of the transdermal patch may comprise 2.0%, 2.5% or 3% w/w oxymorphone.

In another embodiment, the oxymorphone is present in a non-salt form, i.e. as a free base.

### Pharmaceutically Acceptable Pressure Sensitive Polyacrylate Adhesive

The pharmaceutically acceptable pressure sensitive polyacrylate adhesive present in the transdermal patch is selected both in terms of its ability to solubilise oxymorphone, and its adhesive tack and peel properties.

In one embodiment, the pharmaceutically acceptable pressure sensitive polyacrylate adhesive has an opioid analgesic solubility of up to 5% w/w at room temperature. Typically the pharmaceutically acceptable pressure sensitive polyacrylate adhesive has an opioid analgesic solubility of up to 4% w/w at room temperature, most typically, up to 3.5% w/w or 3.0% w/w.

Typically, the total amount of adhesive will constitute between 58 and 99% w/w of the oxymorphone layer of the transdermal patch. Suitably, total amount of adhesive will constitute between 65 and 99% w/w of the oxymorphone layer of the transdermal patch, more suitably between 72 and 97% w/w, yet more suitably between 87 and 95% w/w and most suitably between 92 and 95% w/w.

In an embodiment, the pressure sensitive polyacrylate comprises no functional groups or a plurality of hydroxyl functional groups. Suitably, the pressure sensitive polyacrylate adhesive comprises a plurality of hydroxyl functional groups.

In another embodiment, the pressure sensitive polyacrylate adhesive comprises an acrylate copolymer and/or an acrylate-vinyl acetate. Suitably, the polyacrylate adhesive comprises both an acrylate copolymer and an acrylate-vinyl acetate.

It will be appreciated that the pressure sensitive polyacrylate adhesive of the transdermal patch may comprise a crosslinker.

In an embodiment the pressure sensitive polyacrylate adhesive has a viscosity of between 1600 and 19000 mPa. Suitably, the polyacrylate adhesive has a viscosity of between 4000 and 18000 mPa. More suitably, the polyacrylate adhesive has a viscosity of between 7000 and 18000 mPa. Most suitably, the polyacrylate adhesive has a viscosity of between 7500 and 8500 mPa.

Suitably, the pressure sensitive polyacrylate adhesive is obtained from Henkel.

Any suitable adhesive material or combination of adhesive materials as defined herein may be used.

In an embodiment, the pressure sensitive polyacrylate adhesive comprises a plurality of hydroxyl functional groups and has a viscosity of between 1600 and 19000 mPa. Examples of pressure sensitive polyacrylate adhesives comprising such properties include, but are not limited to, DURO-TAK 87-4287®, DURO-TAK 87-202A®, DURO-TAK 87-2510®, DURO-TAK 87-2287®, DURO-TAK 87-2516® or DURO-TAK 87-2525®, obtainable from Henkel.

In another embodiment, the pressure sensitive polyacrylate adhesive comprises a plurality of hydroxyl functional groups and an acrylate-vinyl acetate, and has a viscosity of between 4000 and 18000 mPa. Examples of pressure sensitive polyacrylate adhesives comprising such properties include, but are not limited to, DURO-TAK 87-4287®, DURO-TAK 87-2287®, DURO-TAK 87-2516® or DURO-TAK 87-2525®.

In another embodiment, the pressure sensitive polyacrylate adhesive comprises a plurality of hydroxyl functional groups and an acrylate-vinyl acetate, has a viscosity of between 7000 and 18000 mPa and is devoid of any crosslinker. Examples of pressure sensitive polyacrylate adhesives comprising such properties include, but are not limited to, DURO-TAK 87-2287® or DURO-TAK 87-4287®.

In a further embodiment, the pressure sensitive polyacrylate adhesive comprises a plurality of hydroxyl functional groups and an acrylate-vinyl acetate, has a viscosity of between 7500 and 8500 mPa and is devoid of any crosslinker. Examples of pressure sensitive polyacrylate adhesives comprising such properties include, but are not limited to DURO-TAK 87-4287®.

Suitably, the pressure sensitive polyacrylate adhesive is selected from DURO-TAK 87-4287®, DURO-TAK 87-2287®, DURO-TAK 87-2516® or DURO-TAK 87-2525®, more suitably, DURO-TAK 87-2287® or DURO-TAK 87-4287®, most suitably, DURO-TAK 87-4287®.

In one embodiment, a suitable volatile solvent is added to the adhesive to reduce viscosity and aid solvation. Suitable solvents may include, but are not limited to, isopropyl alcohol, methanol, ethanol and ethyl acetate.

### Penetration Enhancer

Suitably, the transdermal patch further comprises a penetration enhancer.

The composition may comprise one or more penetration enhancers for transdermal drug delivery. In an embodiment, the penetration enhancer is selected from oleic acid, oleyl alcohol, lauryl alcohol, lauryl acetate, lauryl lactate, ethyl acetate, dimethyl isosorbide, isostearic acid or linoleic acid.

In a particular embodiment, the penetration enhancer is oleic acid or linoleic acid. Suitably, the penetration enhancer is linoleic acid.

In an embodiment, the transdermal patch comprises one penetration enhancer. In another embodiment, the composition comprises two or more penetration enhancers.

The penetration enhancer is present in an amount sufficient to provide the desired physical properties and skin penetration profile for the composition.

For example, one or more pharmaceutically acceptable penetration enhancers can be present in a total amount by weight of 0.1-15% w/w of the oxymorphone layer of the transdermal patch. In an embodiment, one or more pharmaceutically acceptable penetration enhancers are present in a total amount by weight between 2% and 12% w/w of the oxymorphone layer, or between 4% and 10% w/w, or between 4% and 7% w/w, or between 4% and 6% w/w, or between 4.5% and 5.5 w/w, or between 4% and 5% w/w, or about 5% w/w.

### Hydrophilic Materials

In certain embodiments, the use of hydrophilic materials in the transdermal patch may aid the skin absorption of the oxymorphone or the solubility of the oxymorphone in the adhesive. Suitably, the hydrophilic material, and the quantities in which it is added, should be non-toxic, non-irritating, non-allergenic, and compatible with the oxymorphone and the other excipients herein described.

In one embodiment, the hydrophilic material will have a hydrophilic-lipophilic balance (HLB) of greater than 7. Examples of hydrophilic materials suitable for inclusion into the pharmaceutical formulation of the present invention include, but are not limited to, propylene glycol, dipropylene glycol, glycerol, polyethylene glycol, short chain water soluble esters of citric acid, acetic acid, hexylene glycol and alcohols, including diols and polyols.

The amount of hydrophilic material present is 0-20% w/w of the oxymorphone layer of the transdermal patch.

Suitably, when used, the hydrophilic material is present in the transdermal patch in an amount of between 1.0% w/w and 20% w/w of the oxymorphone layer of the transdermal patch.

Suitably, the hydrophilic material, when present, is in an amount of between 0.5 and 10% w/w of the oxymorphone layer of the transdermal patch, and more suitably between 1 and 8% w/w.

Suitably, the hydrophilic material is propylene glycol or dipropylene glycol.

In an embodiment, the hydrophilic material is included in the transdermal patch as part of a mixture including the opioid analgesic, the pharmaceutically acceptable adhesive and a penetration enhancer.

### Overlay

The transdermal patches of the present invention may optionally comprise an overlay layer to help adhere the patch to the skin. The overlay layer may be optionally included for patches with a thickness of greater than about 0.75 mm, where extra adhesion to the skin may be necessary.

It will be appreciated that any suitable overlay (adhesive) may be used to adhere the patch to the skin. Suitably, the overlay is selected from a polyolefin, polyethylene or polyvinyl chloride foam tape.

In an embodiment, the overlay is a foam tape. Suitably, the overlay is a foam tape with one side coated with silicone.

In another embodiment, the overlay is a foam tape with a dry thickness of between 20 and 40 mil (0.51 mm to 1.02 mm). Suitably, the foam tape has a dry thickness of between 28 and 38 mil (0.71 mm to 0.97 mm), and most suitably between 30 and 35 mil (0.76 mm to 0.89 mm).

Suitably, the foam tape is coated with a liner (e.g. polyethylene coated paper). The liner may have any suitable thickness, with the liner typically having a thickness of 0.05% to 0.15% (e.g. 0.10%) of the thickness of the foam tape.

In an embodiment, the liner overlay liner has a thickness of between 2 to 10 mil (51 μm to 0.25 mm), suitably 4 to 6 mil (0.10 mm to 0.15 mm).

In another embodiment, the overlay is obtained from the company 3M.

In a further embodiment, the overlay is selected from 3M CoTran™ 9772 L tape, 3M CoTran™ 9764 tape or 3M CoTran™ 9773 tape.

### Particular Embodiments of the Transdermal Patch

The following represent particular embodiments of the of the oxymorphone layer of the transdermal patch:

| 1.1 | 1-10% | w/w | Oxymorphone; |
|---|---|---|---|
| | 65-98% | w/w | pharmaceutically acceptable pressure sensitive polyacrylate |
| | | | adhesive comprising a plurality of hydroxyl functional groups (e.g. |
| | | | DURO-TAK 87-4287 ®, DURO-TAK 87-2287 ®, DURO-TAK 87- |
| | | | 2516 ® or DURO-TAK 87-2525 ®); |
| | 0.1-15% | w/w | penetration enhancer selected from oleic acid or linoleic acid; |
| | 0-20% | w/w | hydrophilic material (e.g. propylene glycol). |
| 1.2 | 1-6% | w/w | Oxymorphone; |
| | 72-97% | w/w | pharmaceutically acceptable pressure sensitive polyacrylate |
| | | | adhesive comprising a plurality of hydroxyl functional groups (e.g. |
| | | | DURO-TAK 87-4287 ®, DURO-TAK 87-2287 ®, DURO-TAK 87- |
| | | | 2516 ® or DURO-TAK 87-2525 ®); |
| | 2-12% | w/w | penetration enhancer selected from oleic acid or linoleic acid; |
| | 0-10% | w/w | hydrophilic material (e.g. propylene glycol). |
| 1.3 | 1-6% | w/w | Oxymorphone; |
| | 87-95% | w/w | pharmaceutically acceptable pressure sensitive polyacrylate |
| | | | adhesive comprising a plurality of hydroxyl functional groups (e.g. |
| | | | DURO-TAK 87-4287 ®, DURO-TAK 87-2287 ®, DURO-TAK 87- |
| | | | 2516 ® or DURO-TAK 87-2525 ®); |
| | 4-7% | w/w | penetration enhancer selected from oleic acid or linoleic acid. |
| 1.4 | 2-4% w/w | | Oxymorphone; |
| | (e.g. 3% w/w) | | |
| | 91-95% | w/w | pharmaceutically acceptable pressure sensitive polyacrylate |
| | | | adhesive comprising a plurality of hydroxyl functional groups (e.g. |
| | | | DURO-TAK 87-4287 ®, DURO-TAK 87-2287 ®, DURO-TAK 87- |
| | | | 2516 ® or DURO-TAK 87-2525 ®); |
| | 4-5% | w/w | penetration enhancer selected from oleic acid or linoleic acid. |
| 1.5 | 2-3% | w/w | Oxymorphone; |
| | 92-95% | w/w | pharmaceutically acceptable pressure sensitive polyacrylate |
| | | | adhesive comprising a plurality of hydroxyl functional groups, an |
| | | | acrylate-vinyl acetate and a viscosity of between 7000 and 18000 |
| | | | mPa (e.g. DURO-TAK 87-4287); |
| | 4-5% | w/w | penetration enhancer selected from oleic acid or linoleic acid. |

Particular examples of the transdermal patch are provided in the examples defined hereinbelow.

### Permeation Rate of Oxymorphone

It will be understood that the transdermal patch of the present invention, as defined herein, are formulated such that they are capable of administering oxymorphone transdermally during normal use. Suitably, the transdermal patches of the present invention are capable of permeating oxymorphone through the skin (i.e. human skin) during normal use, at any suitable permeation rate (otherwise known as flux).

In an embodiment, the transdermal patches of the present invention have an in vitro human skin permeation rate of oxymorphone that is greater than 1.5 μg cm⁻² h⁻¹. The permeation of oxymorphone through human skin has been measured for selected patches.

Permeation/release measurements of oxymorphone through human male skin (see Examples section hereinbelow) were used as a tool to select candidate patches.

By in vitro human skin permeation rate we mean the rate of delivery of oxymorphone through human skin at time periods up to 72 hours.

Suitably, the in vitro human skin permeation rate of oxymorphone is the apparent steady state flux (calculated from the approximately linear portion of the cumulative permeation profile), typically observed between 3 and 12 hours, or between 24 and 72 hours, when assessed under the conditions detailed in the following sections.

In an embodiment, the in vitro human skin permeation rate of oxymorphone is between 1.5 μg cm⁻² h⁻¹ and 10 μg cm⁻² h⁻¹.

In a further embodiment, the in vitro human skin permeation rate of oxymorphone is between 2.5 μg cm⁻² h⁻¹ and 8 μg cm⁻² h⁻¹.

In a further embodiment, the in vitro human skin permeation rate of oxymorphone is between 3.5 μg cm⁻² Wand 8 μg cm⁻² h⁻¹.

### Combination Patches

In one embodiment, the transdermal patch defined herein containing the oxymorphone can also be combined with an optional second non-opioid pharmacologically active agent for the treatment of pain and/or polydrug abuse, including, for example, a cannabinoid (agonist, antagonist, or inverse agonist), bupropion, hydroxybupropion, nicotine, nornicotine, varenicline, doxepin, acetaminophen, aspirin, diclofenac or another non-steroidal anti-inflammatory drug.

### Therapeutic Uses

The patches of the present invention may be used for the treatment of one or more medical conditions, such as opioid dependence, alcohol dependence, polydrug addiction, pain, cocaine addiction, eating disorders (e.g., binge eating) and treatment-resistant depression are described herein and comprise transdermally administering an opioid from an formulation as defined herein.

The compositions described herein are used in a “pharmacologically effective amount.” This means that the rate and extent of absorption of the active by the subject is such that it results in a therapeutic level of the active in the subject over the period that such compound is to be used. Such delivery is dependent on a number of variables including the time period for which the individual dosage unit is to be used, the flux rate of the active from the composition into the subject, for example, buprenorphine or a buprenorphine prodrug, from the formulation, surface area of the application site, etc.

In another embodiment, a single dosage unit comprises a therapeutically effective amount of oxymorphone. The term “therapeutically effective amount” or “therapeutically and/or prophylactically effective amount” as used herein refers to an amount of oxymorphone that is sufficient to elicit the required or desired therapeutic and/or prophylactic response, as the particular treatment context may require. Single dosage unit as used herein includes individual patches. In one embodiment, a single dosage unit of any formulation comprises a therapeutically effective amount.

It will be understood that a therapeutically effective amount of oxymorphone for a subject is dependent inter alia on the body weight of the subject as well as other factors known to a person of ordinary skill in the art. A “subject” herein to which a therapeutic agent or composition thereof can be administered includes mammals such as a human of either sex and of any age, and also includes any nonhuman animal, particularly a domestic, farm or companion animal, illustratively, a cat, cow, pig, dog or a horse as well as laboratory animals such as guinea pigs and primates.

In one embodiment, compositions described herein are suitable for transdermal administration. In another embodiment, transdermally administrable compositions are adapted for administration to the abdomen, back, chest, legs, arms, scalp or other suitable skin surface.

The terms “treat”, “treated”, “treating” and “treatment” are to be broadly understood as referring to any response to, or anticipation of, a medical condition in a mammal, particularly a human, and includes but is not limited to: (i) inhibiting the medical condition, i.e., arresting, slowing or delaying the on-set, development or progression of the medical condition; or (ii) relieving the medical condition, i.e., causing regression of the medical condition.

In one embodiment, a therapeutically effective amount of oxymorphone is administered transdermally in an formulation as defined herein to treat a medical condition selected from the group consisting of: opioid dependence, alcohol dependence, polydrug addiction, pain, cocaine addiction, eating disorders (e.g., binge eating) and treatment-resistant depression.

Pain can include nociceptive pain, such as somatic pain and visceral pain, and non-nociceptive pain, such as neuropathic pain, sympathetic pain, psychogenic pain and idiopathic pain. Pain also includes chronic and acute pain. Non-limiting examples of pain or sources of pain include fibromyalgia, chronic back pain (both deep and superficial somatic pain), chronic pancreatitis, chronic acute hepatitis, gallstone, appendicitis, post-herpetic neuralgia, trigeminal neuralgia, phantom limb pain, diabetic neuropathy, carpal tunnel syndrome, sciatica, pudendal neuralgia, central pain syndrome, spinal cord injury, post-surgical pain, cancer, degenerative disk disease, osteoporosis, peripheral neuropathy, herpes zoster (shingles), lupus, reflex sympathetic dystrophy, headaches (migraines, tension and cluster), temporomandibular disorders, such as temporomandibular joint syndrome, myofacial pain and internal derangement of the joint and degenerative joint disease, such as osteoarthritis and rheumatoid arthritis.

Eating disorders can include anorexia nervosa, bulimia nervosa, binge eating disorder (BED), compulsive overeating, purging disorder, rumination, diabulimia, food maintenance, eating disorders not otherwise specified (EDNOS), pica, night eating syndrome and orthorexia nervosa.

In one embodiment, the pharmaceutical composition comprising oxymorphone, is administered once daily to a subject in need thereof. In a further embodiment, the pharmaceutical composition comprising oxymorphone, is administered twice daily to a subject in need thereof.

In another illustrative embodiment, a transdermal patch can be one which is capable of controlling the release of oxymorphone or agonist-antagonists or prodrugs of the foregoing such that transdermal delivery of the active compound is substantially uniform and sustained over a period of about 6 hours, about 12 hours, about 24 hours, about 48 hours or about 7 days. Such transdermal patch which can be used in the practice of the methods described herein can take the form of an occlusive body having a backing layer. In practice, the occlusive body which includes the opioid agonists or agonist-antagonists or prodrugs of the foregoing is positioned on the subject's skin under conditions suitable for transdermally delivering the active compound to the subject

### Preparation of Pharmaceutical Formulations

The transdermal patches of the present invention can be prepared using conventional techniques known in the art.

The transdermal patches defined herein are suitably prepared by mixing all of the components together to form a transdermal patch composition, which may then be cast onto a suitable surface (e.g. release liner). The individual components may be mixed by simply adding all of the components at the same time into a mixing vessel and then mixing them all together (a “one-pot” mixture). Alternatively, the components may be added sequentially in two or more steps or stages.

Other experimental conditions required to prepare the formulations of the present invention, such as mixing times, mixing equipment, temperature control etc. can be readily determined by a person of ordinary skill in the art.

In an embodiment, the transdermal patch composition is prepared by the following process:
(i) the pressure sensitive polyacrylate adhesive is mixed with a suitable organic solvent (e.g. ethyl acetate);
(ii) oxymorphone is slowly added to the solution of step (i), with mixing;
(iii) the penetration enhancer is added to the solution of step (ii), with mixing; and
(iv) the solution of step (iii) is further mixed using a roller mixer for at least 8 hours.

Suitably, the mixture if step (iii) is mixed on a roller mixture for at least 10 hours, more suitably, for at least 12 hours, and most suitably for at least 15 hours.

Further experimental details will also be evident from the accompanying Examples.

Once components have been mixed together the transdermal patch can be prepared by wet casting a desired thickness of the transdermal patch composition onto a suitable surface, e.g. a release liner. The transdermal patch can then be dried and stored ready for assembly.

Typically, the transdermal patches are cast at a wet thickness of between 10-60 mil (254 μm to 1.52 mm), to provide a dry thickness of between 4 and 12 mil (102 μm and about 305 μm), suitably between 5 and 11 mil (127 μm and about 279 μm). After casting, the layers are dried.

The transdermal patch may be roller cast onto a suitable surface (e.g. release liner) using any suitable coating gap, i.e. the gap set between rollers. Suitably, the coating gap is set to between 0.100 and 3.00 mm. More suitable, the coating gap is set to between 0.100 and 1.50 mm. Yet more suitably, the coating gap is set to between 0.300 and 1.200 mm. Most suitably, the coating gap is set to between 0.300 and 0.700 mm.

Suitably, the transdermal patch is wet cast onto a release liner as defined herein (e.g. 3M Scotchpak 9744). One surface of the transdermal patch is therefore in contact with the release liner and the backing film is applied to the opposing surface, to complete the assembly of the patch.

### Examples

### DESCRIPTION OF DRAWINGS

Embodiments of the invention will be described, by way of example only, with reference to the accompanying drawings, in which

FIG. 1 shows a plot of the average (n=5) cumulative amount of Oxymorphone (μg/cm²) permeated over time for a 2.5% Oxymorphone transdermal system comprising 2.5% Linoleic Acid (1.2 mm, 10.6 mil, 6.83 mg, 10.75 cm², the patch of Table 4, entry 1 hereinbelow) permeated in vitro through human cadaver skin.

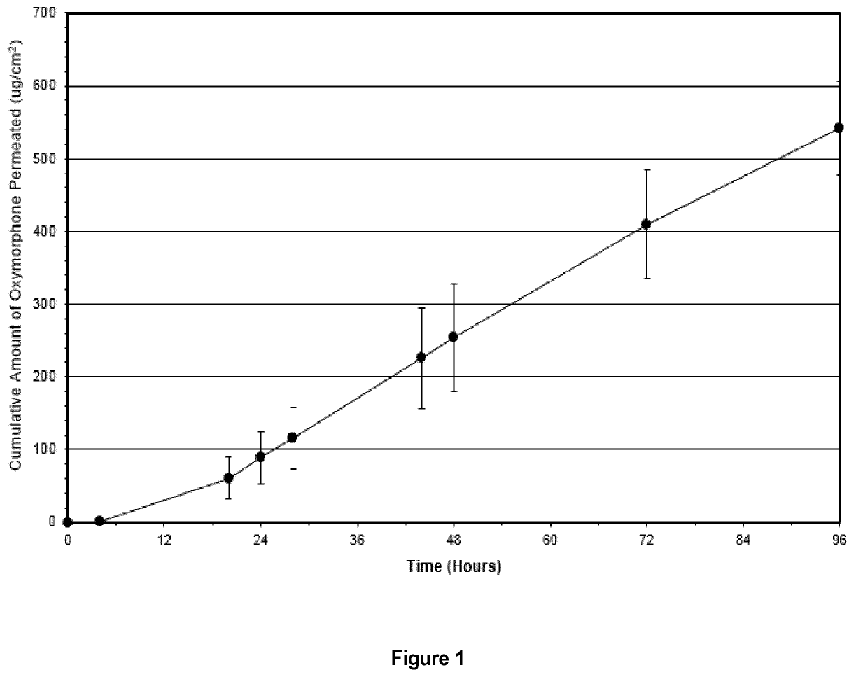

FIG. 2 shows a plot of the average (n=5) Oxymorphone Permeation Rate (μg/cm²/h) over time for a 2.5% Oxymorphone transdermal system comprising 2.5% Linoleic Acid (1.2 mm, 10.6 mil, 6.83 mg, 10.75 cm², the patch of Table 4, entry 1 hereinbelow) permeated in vitro through human cadaver skin.

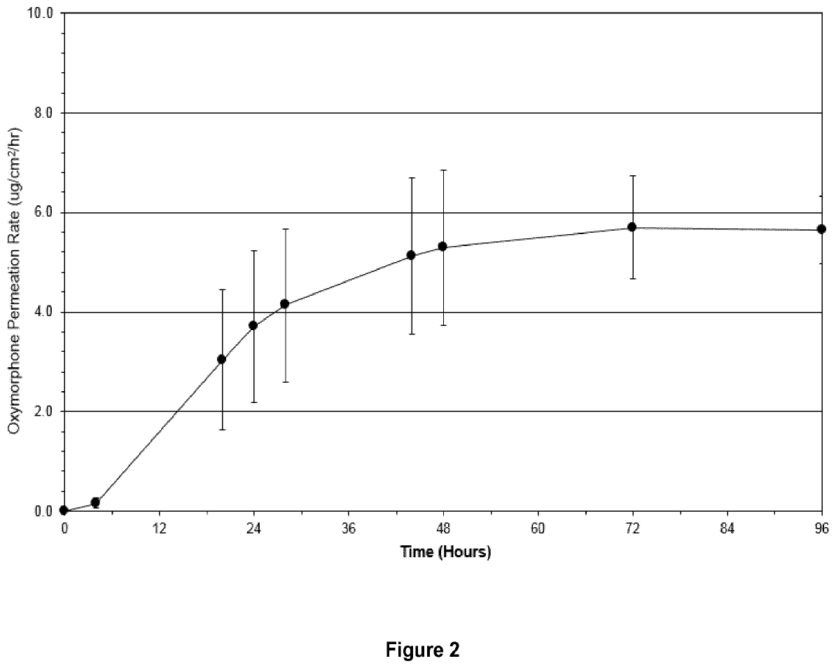

FIG. 3 shows a schematic depicting a typical assembly of a transdermal patch of the present invention (in which: 1 is the backing membrane; 2 is the oxymorphone layer comprising the oxymorphone; and 3 is the release liner).

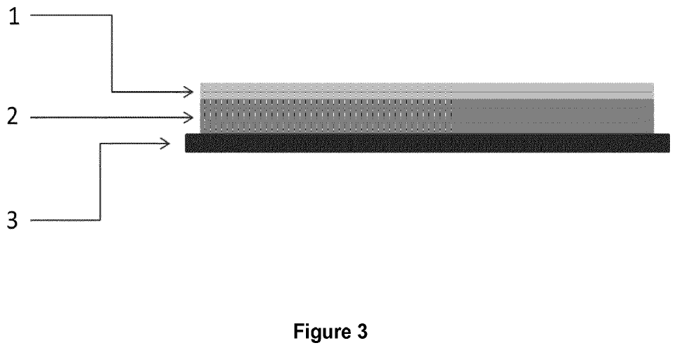

### Materials and Procedures

### Chemicals

The various chemicals used throughout these examples are as follows:

| Chemical | Manufacturer | Lot/Batch | CAS |
|---|---|---|---|
| Oxymorphone base | Mallinckrodt Inc. | 1310000735 | 76-41-5 |
| DURO-TAK 87- | Henkel Corporation | ZQ62566751 | |
| 4287 ® (PSA) | | | |
| Linoleic acid | Spectrum | ZR1141 | 60-33-3 |
| Oleic acid | Croda | 896947 | 112-80-1 |
| Ethyl acetate | | 2DE0179 | 141-78-6 |

### Procedures

### Permeation Studies

### Skin Permeation

Dermatomed/split thickness skin dermatomed to a thickness of about 375 μm from leg (left posterior leg or right posterior leg) was received from Skin Bank. Skin was stored at −5° C. until used for the permeation studies.

### Permeation Studies

Franz Diffusion Cells were used for the skin permeation studies, with the following protocol used.

Diffusion cells were kept at 37° C. with a heated circulating water bath. Skin was cut 3 cm×3 cm and mounted on the top of the receptor of the Franz cells facing stratum corneum/epidermis layer up and dermis layer facing down towards the receptor. The release liner was removed from the transdermal patch (10.5 cm² or 10.75 cm²) and the exposed adhesive layer was applied to the stratum corneum layer with slight pressure. The donor cap was clamped to the receptor compartment. The permeation area of the skin was 1.767 cm². The samples were collected from 3 to 5 cells per formulation. The receptor/receiver solution was PBS (phosphate buffer saline) pH 7.4 solution. Entire samples were withdrawn from the receptor compartment at predetermined time points at 4, 20, 24, 28, 44, 48, 72 and 96 hours and replaced the same volume with fresh PBS solution. The samples were placed screw cap test tubes and refrigerated until ready for HPLC analysis.

### Extraction Studies

### In Ethanol:

Oxymorphone or Naltrexone patches (10.5 cm²) were placed on a polypropylene mesh and placed in a 250 mL glass stoppered conical flask with 100 mL of ethanol. The patches were extracted for 24 hours and the amount of Oxymorphone or Naltrexone was determined, typically by HPLC.

In pH 6.3 buffer:

Oxymorphone or Naltrexone patches (10.5 cm²) were placed on a polypropylene mesh and placed in a 250 mL glass stoppered conical flask with 100 mL of pH 6.3 phosphate buffer. The patches were extracted for 24 hours and the amount of Oxymorphone or Naltrexone was determined, typically by HPLC.

### Results

### Transdermal Patches

### Adhesive

Adhesive transdermal patches were firstly prepared using 3% oxymorphone in various adhesives and determined the corresponding flux values through Male skin age 45 (skin bank JL102114) using Phosphate buffered saline at pH of 7.4. The corresponding flux values are detailed in Table 1 below.

TABLE 1
Flux values of Oxymorphone through Male skin for
transdermal patches comprising various adhesives

| | | | | | | Flux - |
|---|---|---|---|---|---|---|
| Experiment | Adhesive | Adhesive | Functional | Vinyl | Cross | t 24 to 72 |
| Number | Used | Type | Group | Acetate | Linker | μg/cm²/h |
| 1 | DURO-TAK | Polyacrylate | None | VA | None | 2.72 ± 0.42 |
| | 87-4098 | | | Present | | |
| 2 | DURO-TAK | Polyacrylate | Hydroxyl | VA | Present | 4.69 ± 0.44 |
| | 87-2516 | | (—OH) | Present | | |
| 3 | DURO-TAK | Polyacrylate | Carboxyl | VA | None | 0.11 ± 0.03 |
| | 87-2051 | | (—COOH) | Present | | |
| 4 | DURO-TAK | Polyacrylate | Carboxyl | VA | Present | 0.36 ± 0.12 |
| | 87-2054 | | (—COOH) | Present | | |
| 5 | DURO-TAK | Polyacrylate | Hydroxyl | None | None | 2.66 ± 0.42 |
| | 87-2510 | | (—OH) | | | |
| 6 | DURO-TAK | Polyacrylate | None | None | None | 1.65 ± 0.27 |
| | 87-9088 | | | | | |
| 7 | DURO-TAK | Polyacrylate | Carboxyl | VA | Present | 0.15 ± 0.05 |
| | 87-2194 | | (—COOH) | Present | | |
| 8 | DURO-TAK | Polyacrylate | Hydroxyl | VA | None | 3.74 ± 0.23 |
| | 87-4287 | | (—OH) | Present | | |
| 9 | BIO-PSA | Silicone | | | | 0.78 ± 0.11 |
| | 7-4302 | | | | | |
| 10 | Silac | Silicone + | | | | 1.23 ± 0.21 |
| | Hybrid | Polyacrylate | | | | |
| | 7-6302 | | | | | |
| 11 | DURO-TAK | Polyisobutylene | | | | 0.05 ± 0.02 |
| | 87-6918 | | | | | |

Table 1 illustrates the dramatic and unexpected increase in Oxymorphone flux in using polyacrylate adhesives comprising a plurality of hydroxyl functional groups.

### Penetration Enhancer

Adhesive transdermal patches comprising oxymorphone (3% w/w) in DURO-TAK 87-4287 adhesive were prepared using 5 different penetration enhancers (5% w/w) (Oleic acid; oleyl alcohol; oleyl alcohol; lauryl lactate; lauryl alcohol and linoleic acid). Also prepared was a transdermal patch containing oxymorphone (3% w/w) in in DURO-TAK 87-2516 adhesive only. Flux values through Male white skin age 62 (skin bank KMG032615) using Phosphate buffered saline at pH of 7.4 were then determined. The corresponding flux values are in Table 2 below.

It was shown that in all cases, the addition of a penetration enhancer increased Oxymorphone flux through the skin. Penetration enhancers linoleic acid and oleic acid were shown to give substantially increased Oxymorphone flux.

TABLE 2
Flux values of Oxymorphone through Male skin for transdermal
patches comprising various penetration enhancers

| | | | | | | Flux - |
|---|---|---|---|---|---|---|
| Adhesive | Adhesive | Functional | Vinyl | Cross | Penetration | t 24 to 72 |
| Used | Type | Group | Acetate | Linker | Enhancer | μg/cm²/h |
| DURO-TAK | Polyacrylate | Hydroxyl | Present | No | None | 3.15 ± 0.88 |
| 87-4287 | | | | | | |
| DURO-TAK | Polyacrylate | Hydroxyl | Present | No | Oleic Acid | 4.21 ± 0.68 |
| 87-4287 | | | | | | |
| DURO-TAK | Polyacrylate | Hydroxyl | Present | No | Oleyl Alcohol | 2.33 ± 0.66 |
| 87-4287 | | | | | | |
| DURO-TAK | Polyacrylate | Hydroxyl | Present | No | Lauryl Lactate | 2.27 ± 0.48 |
| 87-4287 | | | | | | |
| DURO-TAK | Polyacrylate | Hydroxyl | Present | No | Lauryl Alcohol | 3.53 ± 0.19 |
| 87-4287 | | | | | | |
| DURO-TAK | Polyacrylate | Hydroxyl | Present | No | Linoleic Acid | 3.62 ± 0.76 |
| 87-4287 | | | | | | |

### Flux and Extraction Studies

Oxymorphone flux and extraction in ethanol was determined for the transdermal patches of the present invention. The results are summarised in Table 3 below.

TABLE 3
The extraction and flux data for optimised oxymorphone layer

| | | | OXY FLUX flux |
|---|---|---|---|
| | | | determined using male |
| | Coating | | white skin age 62 |
| Formulation | gap | Extraction Data | Section RPL (μg/cm²/h) |
| 3% oxymorphone in | 0.600 mm | OXYMORPHONE ASSAY | Average flux 24-72 hrs = |
| DURO-TAK 87-4287 | | 1^{st} extraction in ethanol = 4.20 mg | 6.52 |
| and 5% oleic acid | | 2^{nd} extraction in ethanol = 0.12 mg | |
| | | total mg/patch = 4.32 mg | |
| | | 1^{st} extraction in buffer pH 6.3 = 1.72 | |
| | | 2^{nd} extraction in ethanol = 2.17 mg | |
| | | total mg/patch = 3.89 mg | |
| 3% oxymorphone in | 0.600 mm | OXYMORPHONE ASSAY | Average flux 24-72 hrs = |
| DURO-TAK 87-4287 | | 1^{st} extraction in ethanol = 4.15 mg | 6.96 |
| and 5% linoleic acid | | 2^{nd} extraction in ethanol = 0.12 mg | Average flux 0-72 hrs = |
| | | total mg/patch = 4.27 mg | 6.27 |
| | | 1^{st} extraction in buffer pH 6.3 = 1.65 mg | |
| | | 2^{nd} extraction in ethanol = 2.01 mg | |
| | | total mg/patch = 3.66 mg | |

The results in Table 3 show that for the transdermal patches an excellent level of Oxymorphone flux is observed when both linoleic acid and oleic acid are used as the penetration enhancer.

Oxymorphone flux was then determined for the transdermal patches of the present invention at different coating thicknesses. The flux value are summarised in Tables 4 and 5 below.

TABLE 4
Flux values of Oxymorphone for transdermal patches comprising
3% Oxymorphone at different coating thicknesses.

| OXYMORPHONE | | Coating | Permeation | | Flux |
|---|---|---|---|---|---|
| Content | DUROTAK | gap | enhance | Skin Bank donor | (t24-t72 hrs) |
| 3% | 87-4287 | 0.400 | Linoleic | M W (62) | 3.62 +/− 0.76 |
| | | | acid (5%) | MG032615) LPL | |
| 3% | 87-4287 | 0.600 | Linoleic | M W (62) | 6.96 |
| | | | acid (5%) | MG032615 RPL | |
| 3% | 87-4287 | 0.600 | Linoleic | M W (69) | 6.98 |
| | | | acid (5%) | MM041115 PT | |

TABLE 5
Further flux values of Oxymorphone for transdermal patches
comprising 3% Oxymorphone at different coating thicknesses.

| | Amount of | | FLUX VALUE | |
|---|---|---|---|---|
| % | Oxymorphone | COATING | (t 24-72 hrs; | |
| OXYMORPHONE | per cm² | GAP (mm) | units: μg/cm²/h) | COMMENT |
| 3% oxymorphone in | 4.10 mg/10.5 cm² | 5.4 mil | 7.22 (a) | SKIN |
| DURO-TAK 87-4287 | (15.6 mg/40 mg²) | (0.6 mm) | 6.74 (b) | MM040002 |
| and 5% linoleic acid | 390 μg/cm² | | 7.11 (c) | Age 69 |
| | | | 6.88 (d) | section pt |
| | | | 6.83 (e) | |
| | | | Average of 5 | |
| | | | 6.95 μg/cm²/hr | |
| 3% oxymorphone in | 4.24 mg/10.5 cm² | 5.1 mil | 6.81 (a) | SKIN |
| DURO-TAK 87-4287 | (15.77 mg/40 mg²) | (0.6 mm) | 6.72 (b) | JA040115 |
| and 5% linoleic acid | 404 μg/cm² | | 4.03 (c) | Age 67 LPL |
| | | | Average of 3 | |
| | | | 5.86 μg/cm²/hr | |
| 3% oxymorphone in | 5.32 mg/10.75 cm² | 6.3 mil | 7.14 (a) | SKIN |
| DURO-TAK 87-4287 | (19.91 mg/40 mg²) | (0.8 mm) | 6.09 (b) | JA040115 |
| and 5% linoleic acid | 498 μg/cm² | | 7.59 (c) | Age 67 LPL |
| | | | Average of 3 | |
| | | | 6.94 μg/cm²/hr | |
| 3% oxymorphone in | 6.87 mg/10.75 cm² | 8.1 mil | 7.17 (a) | SKIN |
| DURO-TAK 87-4287 | (25.56 mg/40 mg²) | (1.0 mm) | 7.83 (b) | JA040115 |
| and 5% linoleic acid | 639 μg/cm² | | 7.60 (c) | Age 67 LPL |
| | | | Average of 3 | |
| | | | 7.54 μg/cm²/hr | |

### Patches Comprising 2% and 2.5% Oxymorphone

### Flux Values

Using patches comprising 2% and 2.5% Oxymorphone, the flux values of Oxymorphone through male white skin (67 years, skin bank JA040115) were determined. The results are summarised in Table 6 below.

TABLE 6
Flux values of Oxymorphone for transdermal
patches comprising 2% and 2.5% Oxymorphone.

| | Amount of | | FLUX VALUE | |
|---|---|---|---|---|
| % | Oxymorphone | COATING | (t 24-72 hrs; | |
| OXYMORPHONE | per cm² | GAP (mm) | units: μg/cm²/h) | COMMENT |
| 2.5% oxymorphone in | 540 μg/cm² | 0.5 mm + | 7.56 (a) | SKIN |
| DURO-TAK 87-4287 | | 0.5 mm | 7.64 (b) | JA040115 |
| and 5% linoleic acid | | Total = | 7.43 (c) | Age 67 LPL |
| | | 1.0 mm | Average of 3 | |
| | | | 7.54 μg/cm²/hr | |
| 2% oxymorphone in | 434 μg/cm² | 0.5 mm + | 4.31 (a) | SKIN |
| DURO-TAK 87-4287 | | 0.5 mm | 5.22 (b) | JA040115 |
| and 5% linoleic acid | | Total = | 5.35 (c) | Age 67 LPL |
| | | 1.0 mm | Average of 3 | |
| | | | 4.96 μg/cm²/hr | |

### Effects of the Penetration Enhancer

Comparison of patches with and without penetration enhancer were next prepared. The results are summarised in Table 7 below.

TABLE 7
Flux values of Oxymorphone for transdermal patches comprising either no penetration
enhancer or 5% w/w linoleic acid at different coating thicknesses.

| | Amount of | | FLUX VALUE | |
|---|---|---|---|---|
| % | Oxymorphone | COATING | (t 24-72 hrs; | |
| OXYMORPHONE | per cm² | GAP (mm) | units: μg/cm²/h) | COMMENT |
| 3% oxymorphone in | 3.14 mg/10.5 cm² | 3.7 mil (0.4 mm) | 3.85 (a) | SKIN |
| DURO-TAK 87-4287 | (11.96 mg/40 mg²) | | 3.42 (b) | MG032615 |
| | 299 μg/cm² | | 2.16 (c) | Age 62 (white) |
| | | | Average of 3 | section LPL |
| | | | 3.14 μg/cm²/hr | |
| 3% oxymorphone in | 2.93 mg/10.5 cm² | 4.2 mil (0.4 mm) | 2.79 (a) | SKIN |
| DURO-TAK 87-4287 | (11.16 mg/40 mg²) | | 3.78 (b) | MG032615 |
| and 5% linoleic acid | 279 μg/cm² | | 4.28 (c) | Age 62 LPL |
| | | | Average of 3 | |
| | | | 3.62 μg/cm²/hr | |
| 3% oxymorphone in | 4.27 mg/10.5 cm² | 0.6 mm | 7.04 (a) | SKIN |
| DURO-TAK 87-4287 | (16.27 mg/40 mg²) | | 7.74 (b) | MG032615 |
| and 5% linoleic acid | 407 μg/cm² | | 7.07 (c) | Age 62 (white) |
| | | | 6.98 (d) | section RPL |
| | | | 6.48 (e) | |
| | | | Average of 5 | |
| | | | 6.96 μg/cm²/hr | |
| 3% oxymorphone in | 4.12 mg/10.5 cm² | 5.4 mil (0.6 mm) | 7.22 (a) | SKIN |
| DURO-TAK 87-4287 | (15.61 mg/40 mg²) | | 6.74 (b) | MM041115 |
| and 5% linoleic acid | 392 μg/cm² | | 7.11 (c) | Age 69 |
| | | | 6.88 (d) | section pt |
| | | | 6.83 (e) | |
| | | | Average of 5 | |
| | | | 6.95 μg/cm²/hr | |

### Transdermal Patch Prepared by Double Coating Process

A transdermal patch containing a greater amount of Oxymorphone (greater thicknesses of Oxymorphone layer) was prepared and the flux of Oxymorphone through male white skin (69 years, skin bank MM041115) was determined. The patch was made in an identical manner to the other patches described hereinabove, albeit two coatings of the Oxymorphone layer were employed.

The flux values obtained for the ‘thicker’ patches are summarised in Tables 8 and 9 below.

TABLE 8
Flux values of Oxymorphone for a transdermal
patch comprising 2.5% Oxymorphone.

| | Amount | | FLUX VALUE | |
|---|---|---|---|---|
| % | Oxymorphone | COATING | (t 24-72 hrs; | |
| OXYMORPHONE | per cm² | GAP (mm) | units: μg/cm²/h) | COMMENT |
| 2.5% oxymorphone in | 635 μg/cm² | 0.6 mm + | 4.93 (a) | SKIN |
| DURO-TAK 87-4287 | | 0.6 mm | 7.08 (b) | MM041115 |
| and 5% linoleic acid | | Total = | 6.83 (c) | Age 69 LPL |
| | | 1.2 mm | 7.46 (d) | |
| | | | 7.18 (e) | |
| | | | Average of 5 | |
| | | | 6.69 μg/cm²/h | |

TABLE 9

| | Amount | | FLUX VALUE | |
|---|---|---|---|---|
| % | Oxymorphone | COATING | (t 24-72 hrs; | |
| OXYMORPHONE | per cm² | GAP (mm) | units: μg/cm²/h) | COMMENT |
| 2.5% oxymorphone in | 587 μg/cm² | 1.2 mm (10.6 mil) | 5.06 (a) | SKIN |
| DURO-TAK 87-4287 | | | 5.77 (b) | JL080915 |
| and 5% linoleic acid | | | 2.58 (c) | Age 52 RPL |
| | | | Average of 3 | |
| | | | 4.47 μg/cm2/h | |

### Scale-Up Production

A total of 3755×10 cm² transdermal patches were prepared, with each containing 1.72 mg of oxymorphone with a dry weight coating thickness of approximately 2.7 mil (using a coating gap of 0.340 mm).

The flux values for the ‘scale-up’ batch of transdermal patches are provided in Table 10 below.

TABLE 10
Flux values of Oxymorphone for a scale-up transdermal
patch comprising 2.5% Oxymorphone

| | Amount | | FLUX VALUE | |
|---|---|---|---|---|
| % | Oxymorphone | COATING | (t 24-72 hrs; | |
| OXYMORPHONE | per cm² | GAP (mm) | units: μg/cm²/h) | COMMENT |
| 2.5% oxymorphone in | 173 μg/cm² | 0.340 mm | 2.91 (a) | SKIN |
| DURO-TAK 87-4287 | | | 3.17 (b) | CM120115 |
| and 5% linoleic acid | | | 2.98 (c) | Age 48 |
| | | | 2.92 (d) | White Male |
| | | | 2.95 (e) | LPL |
| | | | Average of 5 | |
| | | | 2.99 μg/cm²/h | |

While specific embodiments of the invention have been described for the purpose of reference and illustration, various modifications will be apparent to a person skilled in the art without departing from the scope of the invention as defined by the appended claims.

1. A transdermal patch comprising an oxymorphone layer, which comprises:
1-6% w/w of oxymorphone, wherein the oxymorphone is present in a non-salt form;
82-97% w/w of a pharmaceutically acceptable pressure sensitive polyacrylate adhesive comprising a plurality of hydroxyl functional groups; and
2-12% w/w of a penetration enhancer selected from oleic acid or linoleic acid;
wherein the oxymorphone layer has a first surface that contacts a backing film and a second opposing surface that contacts the skin during use.

2. A transdermal patch according to claim 1, wherein oxymorphone is present at an amount of 2-4% w/w.

3. A transdermal patch according to claim 2, wherein oxymorphone is present at an amount of 2%, 2.5% or 3% w/w.

4. A transdermal patch according to claim 1, wherein the total amount of adhesive will constitute between 87 and 95% w/w.

5. A transdermal patch according to claim 4, wherein the total amount of adhesive will constitute between 92 and 95% w/w.

6. A transdermal patch according to claim 1, wherein the pressure sensitive polyacrylate adhesive has a viscosity of between 1600 and 19000 mPa-s.

7. A transdermal patch according to claim 6, wherein the pressure sensitive polyacrylate adhesive has a viscosity of between 7000 and 18000 mPa-s.

8. A transdermal patch according to claim 6, wherein the pressure sensitive polyacrylate adhesive has a viscosity of between 7500 and 8500 mPa-s.

9. A transdermal patch according to claim 1, wherein the pressure sensitive polyacrylate adhesive comprises a plurality of hydroxyl functional groups and has a viscosity of between 1600 and 19000 mPa-s.

10. A transdermal patch according to claim 9, wherein the pressure sensitive polyacrylate adhesive comprises a plurality of hydroxyl functional groups and an acrylate-vinyl acetate, has a viscosity of between 7500 and 8500 mPa-s and is devoid of any crosslinker.

11. A transdermal patch according to claim 1, wherein the penetration enhancer is linoleic acid.

12. A transdermal patch according to claim 1, wherein the penetration enhancer is present in an amount of 4-6% w/w of the oxymorphone layer.

13. A method of treating a condition selected form the group consisting of opioid dependence, alcohol dependence, polydrug addiction, pain, cocaine addiction, eating disorders and treatment-resistant depression in a subject in need of such treatment, said method comprising administering a transdermal patch according to claim 1 to the skin of the subject.

14. The method of claim 13, wherein the eating disorder is binge eating.
